(19) United States
(12) Patent Application Publication
(10) Pub. No.: US 2004/0192754 A1
(43) Pub. Date: Sep. 30, 2004

(54) Methods for treating idiopathic hyperhidrosis and associated conditions

(76) Inventors: Nathan Andrew Shapira, Gainesville, FL (US); Toby Doris Goldsmith, Gainesville, FL (US); Pierre Martin Blier, Gainesville, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 32606-6669, US

(21) Appl. No.: 10/809,631

(22) Filed: Mar. 24, 2004

Related U.S. Application Data
(60) Provisional application No. 60/457,147, filed on Mar. 24, 2003.

(51) Int. Cl. A61K 31/403

(52) U.S. Cl. 514/412

(57) ABSTRACT

The subject invention provides methods for treating symptoms and/or conditions associated with idiopathic hyperhidrosis by using compounds that decrease the activity of serotonin 5-HT2C receptors. Compounds that can ameliorate symptoms of idiopathic hyperhidrosis and associated conditions include 5-HT2C receptor antagonists (i.e., ketanserin, ritanserin, mianserin, mesulergine, cyproheptadine, fluoxetine, mirtazapine, olanzapine, and ziprasidone) as well as 5-HT2C receptor modulators (i.e., inverse agonists, partial agonists, and allosteric modulators).

### CROSS-REFERENCE TO RELATED APPLICATION

This application claims the benefit of U.S. Provisional Application No. 60/457,147, filed Mar. 24, 2003.

### BACKGROUND OF INVENTION

Sweating is a physiological response to heat which affords protective evaporative cooling through the skin. Sweating in excess of what is required for thermoregulation by exocrine sweat glands is called hyperhidrosis. These glands, while present over the entire body surface, are most concentrated on axillae, face, palms, and soles followed by back and chest.

Hyperhidrosis can be localized or generalized. While generally considered non-life-threatening, hyperhidrosis can cause emotional distress and social embarrassment as well as destruction of private and professional lives and affects. While almost everyone has had at least one episode of excessive sweating in their lives, most disabling hyperhidrosis is estimated to affect 0.6% to 1.0% of the population. The incidence is highest among infants, teenagers, and young adults and occurs equally in both sexes, although females may be more distressed and present for treatment more than males. Hyperhidrosis may be idiopathic/essential (designating a disease having no known cause) or secondary to other diseases, metabolic disorders, febrile illnesses, and drugs (i.e., an iatrogenic event or complication).

There are multiple skin conditions which can be predisposed by hyperhidrosis including trench foot, ingrown nails, pitted keratolysis, and frostbite (due to accumulation of moisture in shoes in cold environments). Hyperhidrosis can lead to heat stroke if prolonged due to loss of electrolytes and fluid. Hyperhidrosis can aggravate exzematous dermatitis and can place individuals at risk for contact dermatitis and miliaria. Hyperhidrosis, particularly of the feet, can encourage mycotic, bacterial, and viral lesion growth and is commonly associated with bromhidroses, commonly known as body odor, and its treatment can facilitate improvement of these growths and reduction in bromhidroses.

Current treatments for hyperhidrosis are symptomatic unless the physiological factor or condition causing the hyperhidrosis is known. One form of treatment for idiopathic hyperhidrosis is the systemic use of anti-cholinergic compounds. This form of treatment is often limited due to transient benefits and adverse side effects. Other forms of treatment include local administration of botulinum toxin or surgical treatments. Unfortunately, botulinum toxin treatments are expensive and, due to its nature, surgery is generally performed only as a last resort. Therefore, there is a current need for an effective medication which, when used alone or in combination with other treatments for hyperhidrosis, can ameliorate symptoms of idiopathic hyperhidrosis and its associated conditions.

Receptors for serotonin (5-hydroxytryptamine) are termed serotonin or 5-HT receptors. The 5-HT2 receptor belongs to the family of rhodopsin-like signal transducers, which are distinguished by their seven-transmembrane configuration and their functional linkage to G-proteins. While all the receptors of the serotonin type recognize serotonin, they are pharmacologically distinct and are encoded by separate genes. These receptor subtypes are generally coupled to different second messenger pathways that are linked through guanine-nucleotide regulatory (G) proteins. Among the serotonin receptors, 5-HT1A, 5-HT1B, and 5-HT1D receptors inhibit adenylate cyclase, and 5-HT2A and 5-HT2C receptors activate phospholipase C pathways, stimulating breakdown of polyphosphoinositides. Theoretically, dysfunctions of the serotonin 5-HT2C receptors, including alterations in receptor number, function, or interactions of these receptors with other systems, may play an important role in idiopathic hyperhidrosis.

### BRIEF SUMMARY

The subject invention provides materials and methods for treating symptoms and/or conditions associated with idiopathic hyperhidrosis and/or sweating by using compounds that decrease the activity of serotonin 5-HT2C receptors. Compounds that can ameliorate symptoms of idiopathic hyperhidrosis and associated conditions according to the subject invention include 5-HT2C receptor antagonists as well as 5-HT2C receptor modulators. 5-HT2C receptor antagonists specifically exemplified herein include ketanserin, ritanserin, mianserin, mesulergine, cyproheptadine, fluoxetine, mirtazapine, olanzapine, and ziprasidone. 5-HT2C receptor modulators include, but are not limited to, inverse agonists, partial agonists, and allosteric modulators of 5-HT2C receptors.

In one embodiment of the present invention, therapeutically effective amounts, of a compound that decreases the activity of serotonin 5-HT2C receptors is administered to a patient with idiopathic hyperhidrosis to alleviate and/or treat symptoms of hyperhidrosis and/or the condition itself.

In another embodiment, therapeutically effective amounts of a 5-HT2C receptor activity affecting compound is administered to a patient prior to exposure to a situation and/or environment known to cause sweating by the patient. For example, in accordance with the subject application, a 5-HT2C receptor activity affecting compound can be administered to a patient prior to exposure to hot air temperatures.

### DETAILED DISCLOSURE

The subject invention pertains to the treatment of symptoms or associated conditions of idiopathic hyperhidrosis. Methods for treating symptoms and conditions associated with idiopathic hyperhidrosis are provided using compounds that decrease the activity of serotonin receptors. In a preferred embodiment, therapeutic amounts of at least one compound that affects the activity of 5-HT2C receptors is administered to treat symptoms or associated conditions of idiopathic hyperhidrosis.

The subject invention also provides methods for prophylactically preventing or minimizing sweat secretion on a patient's skin, especially in axillary (underarm) regions, as a result of perspiring. In one embodiment, therapeutic amounts of at least one compound that affects the activity of 5-HT2C receptors is administered to a patient prior to exposure to condition that is known to induce sweating (i.e., hot temperature, physical activity, increased sympathetic nerve activity as a result of emotional state (i.e., job interview, oral presentation)) to prevent or minimize sweating.

The term “hyperhidrosis” or “idiopathic hyperhidrosis,” as used herein, refers to a commonly known medical condition having no associated disease or cause, which is characterized by excessive, uncontrollable perspiration beyond that required to cool the body. For example, idiopathic hyperhidrosis is often characterized as excessive sweating, usually on the palms of the hand, soles of the feet, or armpit areas, that is not caused by emotional or physical activity.

“Sweating” or “perspiring,” as used herein, refers to the biological act of fluid secretion by the ecrrine and/or apocrine glands in a patient in response to nerve stimulation, emotional state, environmental conditions (i.e., hot air temperature), and/or exercise.

The term “therapeutically effective amount,” as used herein, refers to that amount of a drug or pharmaceutical agent that will elicit the biological or medical response of a tissue, system, animal, or human that is being sought by a researcher, veterinarian, medical doctor, or clinician. In particular, with regard to treating those conditions or symptoms associated with hyperhidrosis, a “therapeutically effective amount” is intended to mean that amount of 5-HT2C receptor activity affecting compound that will prevent or alleviate those conditions or symptoms.

The term “5-HT2C receptor activity affecting compound,” as used herein, refers to those compounds that can decrease serotonin 5-HT2C receptor activity. Contemplated 5-HT2C receptor activity affecting compounds include 5-HT2C receptor antagonists (i.e., ketanserin, ritanserin, mianserin, mesulergine, cyproheptadine, fluoxetine, mirtazapine, olanzapine, and ziprasidone) as well as 5-HT2C receptor modulators (i.e., inverse agonists, partial agonists, and allosteric modulators).

The 5-HT2C receptor activity affecting compounds of the present invention may have chiral centers, and therefore may occur as racemates, racemic mixtures, and as individual enantiomers or diastereomers, with all such isomeric forms being included in the present invention as well as mixtures thereof. Furthermore, some of the crystalline forms for the 5-HT2C receptor activity affecting compounds of the present invention may exist as polymorphs and as such are intended to be included in the present invention. In addition, some of the 5-HT2C receptor activity affecting compounds of the instant invention may form solvates with water or common organic solvents. Such solvates are encompassed within the scope of this invention.

The subject invention provides methods having both human and veterinary utility. The term “individual” or “patient” includes animals of avian, mammalian, or reptilian origin. Mammalian species that benefit from the disclosed methods include, and are not limited to, apes, chimpanzees, orangutans, humans, monkeys, dogs, cats, guinea pigs, and mice.

In one embodiment, a 5-HT2C receptor activity affecting compound is administered alone to patients diagnosed with idiopathic hyperhidrosis to treat associated systems or conditions. In another embodiment, a 5-HT2C receptor activity affecting compound is administered concurrently with other agents commonly used in preventing sweating to ameliorate symptoms of idiopathic hyperhidrosis. A further embodiment provides administering a 5-HT2C receptor activity affecting compound, either alone or concurrently with other agents commonly used in preventing sweating, to a patient prior to exposure to condition that is known to induce sweating (i.e., hot temperature, physical activity, increased sympathetic nerve activity as a result of emotional state (i.e., job interview, oral presentation)) to prophylactically prevent or minimize sweating.

“Administered concurrently” and “concurrently administering,” as used herein, includes administering a compound or therapeutic method suitable for use with the methods of the invention (administration of a 5-HT2C receptor activity affecting compound) in the treatment of idiopathic hyperhidrosis and/or symptoms or associated conditions of idiopathic hyperhidrosis. For example, a 5-HT2C receptor activity affecting compound can be administered concurrently with agents such as antiperspirants (i.e., aluminum metal salts), compounds commonly used to block acetylcholine from stimulating sweat glands, also referred to herein as acetylcholine-blocking compounds (i.e., anticholinergics, antihistamines, antidepressants, tranquilizers), and beta blockers. Specific agents that can be administered concurrently with a 5-HT2C receptor activity affecting compound include, without limitation, aluminum acetate, aluminum sulfate, aluminum chloride, propranolol, glycopyrrolate, atropine, propantheline bromide, and oxybutynin.

According to the present invention, a 5-HT2C receptor activity affecting compound can be administered concurrently with known methods for treating sweating including, without limitation, iontophoresis (which includes the “injection” of electrically charged ions into the skin, which interacts with the sweat glands and ducts to cause them to stop secreting sweat), endoscopic thoracic sympathicotomy, and injection of botulinum toxin.

By way of example, an agent can be provided in admixture with a 5-HT2C receptor activity affecting compound, such as in a pharmaceutical composition; or the agent and 5-HT2C receptor activity affecting compound can be provided as separate compounds, such as, for example, separate pharmaceutical compositions administered consecutively, simultaneously, or at different times. Preferably, if the 5-HT2C receptor activity affecting compound and the known agent (or therapeutic method) for treating idiopathic hyperhidrosis are administered separately, they are not administered so distant in time from each other that the 5-HT2C receptor activity affecting compound and the known agent (or method) cannot interact.

Contemplated 5-HT2C receptor activity affecting compounds of the present invention include (1R,2S,4R)-(−)-2-phenyl 2-(dimethylaminoethoxy)-1,7,7-trimethyl-bicyclo[2.2.1]heptane, known as deramciclane, and (1R,2S,4R)-(−)-2-phenyl-2-(methylaminoethoxy)-1,7,7-trimethyl-bicyclo[2.2.1]heptane, and their pharmaceutically acceptable acid addition salts with inorganic and organic acids, are taught and disclosed in U.S. Pat. No. 4,342,762 and International Patent Application No. WO 98/17230, respectively, which are both incorporated herein by reference. These compounds are selective serotonin 5-HT2C receptor antagonists.

Another contemplated 5-HT2C receptor activity affecting compound of the present invention is mirtazapine, which is disclosed in U.S. Pat. No. 4,062,848. The present invention includes the use of any particular enantiomer alone, or in a mixture with one or more stereoisomers, in any proportion including racemic mixtures of mirtazapine. Further, the present invention includes any salts of the compound, such as acid addition salts, for example, hydrochloric, fumaric, maleic, citric or succinic acid, these acids being mentioned only by way of illustration and without implied limitation. These compounds can be prepared in accordance with U.S. Pat. No. 4,062,848, incorporated herein by reference.

Other 5-HT2C receptor activity affecting compounds of the present invention include those compounds disclosed in U.S. Pat. No. 6,420,541. These compounds, also known as modulators, have demonstrated inverse agonist characteristics at serotonin 5-HT2C receptors.

In the present invention, the 5-HT2C receptor activity affecting compounds form the active ingredient for ameliorating the symptoms or associated conditions of idiopathic hyperhidrosis or for prophylactically preventing sweating. These compounds are typically administered in admixture with suitable pharmaceutical diluents, excipients, and/or carriers (collectively referred to as “carrier” materials) suitably selected with respect to the intended form of administration. The term “excipients,” as used herein, refers to compositions that retain the biological effectiveness and properties of the 5-HT2C receptor activity affecting compounds of this invention and which are not biologically or otherwise undesirable for administration to a patient. 5-HT2C receptor activity affecting compounds, in accordance with the present invention, can be administered orally (alimentary), via mucosa, systemically, topically, parenterally (i.e., intravenous, including both bolus and infusion, intraperitoneal, subcutaneous, and/or intramuscular), formulations of which are known to those of ordinary skill in the pharmaceutical arts. For example, suitable routes of administration that can be employed for providing the patient with a therapeutically effective amount of 5-HT2C receptor activity affecting compound include intraoral, rectal, epicutaneous, transdermal, intranasal, sublingual, buccal, intradural, intraocular, intrarespiratory, or nasal inhalation and like forms of administration.

Suitable forms for topical administration include, but are not limited to, dispersions, lotions; creams; gels; pastes; powders; aerosol sprays; syrups or ointments on sponges or cotton applicators; and solutions or suspensions in an aqueous liquid, non-aqueous liquid, oil-in-water emulsion, or water-in-oil liquid emulsion. Because of its ease of administration, a cream, lotion, or ointment represents the most advantageous topical dosage unit form, in which case liquid pharmaceutical carriers may be employed in the composition. These creams, lotions, or ointments, may be prepared as rinse-off or leave-on products, as well as two stage treatment products for use with other skin cleansing or managing compositions. Each of these forms is well understood by those of ordinary skill in the art, such that dosages may be easily prepared to incorporate the 5-HT2C receptor activity affecting compound of the invention.

Suitable pharmaceutical formulations can be administered in a variety of forms including, for example, tablets, capsules (each including timed release and sustained release formulations), pills, powders, granules, elixirs, tinctures, solutions, suspensions, syrups, emulsions. Preferably, 5-HT2C receptor activity affecting compounds are administered orally.

In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like.

Because of their ease in administration, tablets and capsules represent the most advantageous oral dosage unit form, in which case solid pharmaceutical carriers are employed. If desired, tablets may be sugar coated or enteric coated by standard techniques. Suppositories may be prepared, in which case cocoa butter could be used as the carrier.

For parenterals, the carrier will usually comprise sterile water, though other ingredients, for example, for purposes such as aiding solubility or for preservation, may be included. Injectable suspensions may also be prepared in which case appropriate liquid carriers, suspending agents and the like may be employed.

The dosage regimen utilizing the compounds of the present invention is selected in accordance with a variety of factors including, type, species, age, weight, sex, and medical condition of the patient; the severity of the condition to be treated, the route of administration; the renal and hepatic function of the patient; and the particular compound thereof employed. A physician or veterinarian of ordinary skill can readily determine and prescribe the therapeutically effective amount of a 5-HT2C receptor activity affecting compound required to prevent, counter, or arrest the progress of the condition or symptom associated with idiopathic hyperhidrosis. Optimal precision in achieving concentration of drug within the range that yields efficacy without toxicity requires a regimen based on the kinetics of the drugs availability to target 5-HT2C receptor sites. This involves a consideration of the distribution, equilibrium, and elimination of the drug.

In one embodiment, the 5-HT2C receptor activity affecting compound is mirtazapine. It is contemplated herein that the useful dosage of mirtazapine for use in the method of the present invention ranges from 0.5 to 1000 mg per adult human per day. Preferably, dosages range from 1 to 200 mg/day. More preferably, dosages range from 5-50 mg/day. Advantageously, in accordance with the present invention, mirtazapine may be administered in a single daily dose, or the total daily dosage may be administered in dividend doses (i.e., two, three or four times daily).

In another embodiment, the 5-HT2C receptor activity affecting compound is olanzapine. It is contemplated herein that the useful dosage of olanzapine for use in the method of the present invention ranges from 0.5 to 1000 mg per adult human per day. Preferably, dosages range from 1 to 100 mg/day. More preferably, dosages range from 5-50 mg/day. Advantageously, in accordance with the present invention, olanzapine may be administered in a single daily dose, or the total daily dosage may be administered in dividend doses (i.e., two, three or four times daily).

In yet another embodiment, the 5-HT2C receptor activity affecting compound is cyproheptadine. It is contemplated herein that the useful dosage of cyproheptadine for use in the method of the present invention ranges from 0.5 to 1000 mg per adult human per day. Preferably, dosages range from 1 to 200 mg/day. More preferably, dosages range from 5-50 mg/day. Advantageously, in accordance with the present invention, cyproheptadine may be administered in a single daily dose, or the total daily dosage may be administered in dividend doses (i.e., two, three or four times daily).

In another embodiment, the 5-HT2C receptor activity affecting compound is fluoxetine. It is contemplated herein that the useful dosage of fluoxetine for use in the method of the present invention ranges from 0.5 to 1000 mg per adult human per day. Preferably, dosages range from 1 to 200 mg/day. More preferably, dosages range from 5-100 mg/day. Advantageously, in accordance with the present invention, fluoxetine may be administered in a single daily dose, or the total daily dosage may be administered in dividend doses (i.e., two, three or four times daily).

In a further embodiment, the present invention provides the administration of at least one compound that decreases the activity at 5-HT2C receptor sites in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for treating idiopathic hyperhidrosis, wherein said method comprises administering to a patient a therapeutically effective amount of a 5-HT2C receptor activity affecting compound.

2. The method of claim 1, wherein said 5-HT2C receptor activity affecting compound is selected from the group consisting of 5-HT2C receptor antagonists and 5-HT2C modulators.

3. The method of claim 2, wherein said 5-HT2C receptor antagonist is selected from the group consisting of ketanserin, ritanserin, mianserin, meulergine, cyproheptadine, fluoxetine, mirtazapine, olanzapine, and ziprasidone.

4. The method of claim 2, wherein said 5-HT2C modulator is selected from the group consisting of inverse agonists, partial agonists, and allosteric modulators.

5. The method of claim 1, wherein said 5-HT2C receptor activity affecting compound is selected from the group consisting of (1R,2S,4R)-(−)-2-phenyl 2-(dimethylaminoethoxy)-1,7,7-trimethyl-bicyclo[2.2.1]heptane and (1R,2S,4R)-(−)-2-phenyl-2-(methylaminoethoxy)-1,7,7-trimethyl-bicyclo[2.2.1]heptane.

6. The method of claim 1, wherein said 5-HT2C receptor activity affecting compound is administered to the patient via a route selected from the group consisting of oral, topical, mucosal, systemic, parenteral, intravenous, intraperitoneal, subcutaneous, intramuscular, intraoral, rectal, epicutaneous, transdermal, intranasal, sublingual, buccal, intradural, intraocular, intrarespiratory, and intra nasal inhalation.

7. The method of claim 1, wherein said 5-HT2C receptor activity affecting compound is administered to the patient via liposome delivery systems.

8. The method of claim 1, further comprising the step of concurrently administering an agent used to treat sweating.

9. The method of claim 8, wherein said agent is selected from the group consisting of antiperspirants, acetylcholine-blocking compounds, and beta blockers.

10. The method of claim 9, wherein said agent is selected from the group consisting of aluminum acetate, aluminum sulfate, aluminum chloride, propranolol, glycopyrrolate, atropine, propantheline bromide, and oxybutynin.

11. The method of claim 1, further comprising the step of concurrently administering a method for treating sweating.

12. The method of claim 11, wherein said method for treating sweating is selected from the group consisting of iontophoresis, endoscopic thoracic sympathicotomy, and botulinum toxin injection.

13. A method for treating symptoms or associated conditions of idiopathic hyperhidrosis, wherein said method comprises administering to a patient a therapeutically effective amount of a 5-HT2C receptor activity affecting compound.

14. The method of claim 13, wherein said 5-HT2C receptor activity affecting compound is selected from the group consisting of 5-HT2C receptor antagonists and 5-HT2C modulators.

15. The method of claim 14, wherein said 5-HT2C receptor antagonist is selected from the group consisting of ketanserin, ritanserin, mianserin, meulergine, cyproheptadine, fluoxetine, mirtazapine, olanzapine, and ziprasidone.

16. The method of claim 14, wherein said 5-HT2C modulator is selected from the group consisting of inverse agonists, partial agonists, and allosteric modulators.

17. The method of claim 13, wherein said 5-HT2C receptor activity affecting compound is selected from the group consisting of (1R,2S,4R)-(−)-2-phenyl 2-(dimethylaminoethoxy)-1,7,7-trimethyl-bicyclo[2.2.1]heptane and (1R,2S,4R)-(−)-2-phenyl-2-(methylaminoethoxy)-1,7,7-trimethyl-bicyclo[2.2.1]heptane.

18. The method of claim 13, wherein said 5-HT2C receptor activity affecting compound is administered to the patient via a route selected from the group consisting of oral, topical, mucosal, systemic, parenteral, intravenous, intraperitoneal, subcutaneous, intramuscular, intraoral, rectal, epicutaneous, transdermal, intranasal, sublingual, buccal, intradural, intraocular, intrarespiratory, and intra nasal inhalation forms.

19. The method of claim 13, wherein said 5-HT2C receptor activity affecting compound is administered to the patient via liposome delivery systems.

20. The method of claim 13, further comprising the step of concurrently administering an agent used to treat sweating.

21. The method of claim 20, wherein said agent is selected from the group consisting of antiperspirants, acetylcholine-blocking compounds, and beta blockers.

22. The method of claim 21, wherein said agent is selected from the group consisting of aluminum acetate, aluminum sulfate, aluminum chloride, propranolol, glycopyrrolate, atropine, propantheline bromide, and oxybutynin.

23. The method of claim 13, further comprising the step of concurrently administering a method for treating sweating.

24. The method of claim 23, wherein said method for treating sweating is selected from the group consisting of iontophoresis, endoscopic thoracic sympathicotomy, and botulinum toxin injection.

25. A composition comprising a therapeutically effective amount of a 5-HT2C receptor activity affecting compound for treating idiopathic hyperhidrosis and an agent used to treat sweating.

26. The composition of claim 25, wherein said agent is selected from the group consisting of antiperspirants, acetylcholine-blocking compounds, and beta blockers.

27. The composition of claim 26, wherein said agent is selected from the group consisting of aluminum acetate, aluminum sulfate, aluminum chloride, propranolol, glycopyrrolate, atropine, propantheline bromide, and oxybutynin.

28. The composition of claim 25, wherein said 5-HT2C receptor activity affecting compound is selected from the group consisting of 5-HT2C receptor antagonists and 5-HT2C modulators.

29. The composition of claim 28, wherein said 5-HT2C receptor antagonist is selected from the group consisting of ketanserin, ritanserin, mianserin, meulergine, cyproheptadine, fluoxetine, mirtazapine, olanzapine, and ziprasidone.

30. The composition of claim 28, wherein said 5-HT2C modulator is selected from the group consisting of inverse agonists, partial agonists, and allosteric modulators.

31. The composition of claim 25, wherein said 5-HT2C receptor activity affecting compound is selected from the group consisting of (1R,2S,4R)-(−)-2-phenyl 2-(dimethylaminoethoxy)-1,7,7-trimethyl-bicyclo[2.2.1]heptane and (1R,2S,4R)-(−)-2-phenyl-2-(methylaminoethoxy)-1,7,7-trimethyl-bicyclo[2.2.1]heptane.

32. A method for prophylactically preventing or minimizing perspiring, wherein said method comprises administering to a patient a therapeutically effective amount of a 5-HT2C receptor activity affecting compound.

33. The method of claim 32, wherein said 5-HT2C receptor activity affecting compound is selected from the group consisting of 5-HT2C receptor antagonists and 5-HT2C modulators.

34. The method of claim 33, wherein said 5-HT2C receptor antagonist is selected from the group consisting of ketanserin, ritanserin, mianserin, meulergine, cyproheptadine, fluoxetine, mirtazapine, olanzapine, and ziprasidone.

35. The method of claim 33, wherein said 5-HT2C modulator is selected from the group consisting of inverse agonists, partial agonists, and allosteric modulators.

36. The method of claim 32, wherein said 5-HT2C receptor activity affecting compound is selected from the group consisting of (1R,2S,4R)-(−)-2-phenyl 2-(dimethylaminoethoxy)-1,7,7-trimethyl-bicyclo[2.2.1]heptane and (1R,2S,4R)-(−)-2-phenyl-2-(methylaminoethoxy)-1,7,7-trimethyl-bicyclo[2.2.1]heptane.

37. The method of claim 32, wherein said 5-HT2C receptor activity affecting compound is administered to the patient via a route selected from the group consisting of oral, topical, mucosal, systemic, parenteral, intravenous, intraperitoneal, subcutaneous, intramuscular, intraoral, rectal, epicutaneous, transdermal, intranasal, sublingual, buccal, intradural, intraocular, intrarespiratory, and intra nasal inhalation forms.

38. The method of claim 32, wherein said 5-HT2C receptor activity affecting compound is administered to the patient via liposome delivery systems.

39. The method of claim 32, further comprising the step of concurrently administering an agent used to treat sweating.

40. The method of claim 39, wherein said agent is selected from the group consisting of antiperspirants, acetylcholine-blocking compounds, and beta blockers.

41. The method of claim 40, wherein said agent is selected from the group consisting of aluminum acetate, aluminum sulfate, aluminum chloride, propranolol, glycopyrrolate, atropine, propantheline bromide, and oxybutynin.

42. The method of claim 32, further comprising the step of concurrently administering a method for treating sweating.

43. The method of claim 32, wherein said method for treating sweating is selected from the group consisting of iontophoresis, endoscopic thoracic sympathicotomy, and botulinum toxin injection.
